(19) United States
(12) Patent Application Publication
(10) Pub. No.: US 2026/0021259 A1
(43) Pub. Date: Jan. 22, 2026

(54) NEBULIZER SPRAY CHAMBER ASSEMBLY

(71) Applicant: Waters Technologies Corporation, Milford, MA (US)

(72) Inventor: Joseph A. Jarrell, Newton Highlands, MA (US)

(21) Appl. No.: 19/273,970

(22) Filed: Jul. 18, 2025

Related U.S. Application Data
(60) Provisional application No. 63/673,028, filed on Jul. 18, 2024.

(51) Int. Cl. A61M 11/06; A61M 11/00

(52) U.S. Cl. CPC A61M 11/06; A61M 11/001

(57) ABSTRACT

A nebulizer spray chamber assembly lacks a horizontally extending rib or partition. The lack of the horizontally extending rib or partition prevents droplets in a spray chamber from falling into a plume. The nebulizer spray chamber assembly has an exit oriented in a direction that an aerosol plume is introduced into the spray chamber (i.e., a primary horizontal direction). Since there is no change in direction of gas flow toward the nebulizer nozzle, more droplets may be detected by a detector, such as a charged aerosol detector.

### CROSS-REFERENCE TO RELATED APPLICATIONS

This application claims the benefit of and priority to U.S. Provisional Patent Application No. 63/673,028 filed Jul. 18, 2024, the entire contents of which are hereby incorporated by reference.

### BACKGROUND

A nebulizer is a piece of equipment that produces a fine spray from a liquid. Nebulizers are used in a variety of types of conventional devices. One example of a device where a nebulizer is used is a charged aerosol detector (CAD), which measures amounts of components in a sample.

### SUMMARY

In accordance with an inventive facet, a nebulizer and spray chamber assembly includes a nebulizer for producing a droplet spray from a liquid sample stream and a gas stream. The nebulizer and spray chamber assembly further includes a spray chamber having an upper portion, a central portion, and a lower portion. The nebulizer introduces the droplet spray into the central portion of the spray chamber from an entry point in a primary horizontal direction of travel towards a wall of the central portion of the spray chamber that is substantially perpendicular to the primary horizontal direction of travel of the droplet spray. The upper portion is positioned above the entry point and is connected to the central portion of the spray chamber to receive gas flow and entrained droplets flowing from the central portion. The lower portion is positioned below the entry point and is connected to the central portion of the spray chamber for receiving accumulated liquid in the spray chamber. The nebulizer and spray chamber assembly includes an exit positioned in the upper chamber at a position that is horizontally offset relative to the entry point in the primary horizontal direction of travel for gas flow and entrained droplets to leave the spray chamber.

The nebulizer may include an emitter nozzle. The emitter nozzle may be connected to a wall of the central portion of the spray chamber. The nebulizer and spray chamber assembly may include a drain positioned in the lower portion of the spray chamber for draining the accumulated liquid. The lower portion of the spray chamber may include a curved wall for directing the accumulated liquid to the drain. The upper portion of the spray chamber may include a curved wall for directing the gas flow and entrained droplets to the exit. The nebulizer may be a concentric pneumatic nebulizer.

In accordance with another inventive facet, a nebulizer and spray chamber assembly includes a nebulizer for producing a droplet spray from a liquid sample stream and a gas stream and a spray chamber having an upper portion, a central portion and a lower portion. The nebulizer introduces the droplet spray into the central portion of the spray chamber from an entry point in a primary horizontal direction of travel towards a wall of the central portion of the spray chamber that is oriented at an obtuse angle relative to the primary horizontal direction of travel of the droplet spray. The upper portion is positioned above the entry point and is connected to the central portion of the spray chamber to receive gas flow and entrained droplets flowing from the central portion. The lower portion is positioned below the entry point and is connected to the central portion of the spray chamber for receiving accumulated liquid in the spray chamber. The nebulizer and spray chamber assembly further includes an exit positioned in the upper chamber at a position that is horizontally offset relative to the entry point in primary horizontal direction of travel for gas flow and entrained droplets to leave the spray chamber.

The nebulizer may include an emitter nozzle. The emitter nozzle may be connected to a wall of the central portion of the spray chamber. The nebulizer and spray chamber assembly may include a drain positioned in the lower portion of the spray chamber for draining the accumulated liquid. The upper portion of the spray chamber may include an extension that is an extension of the wall of the central portion of the spray chamber and that is at the obtuse angle relative to the primary horizontal direction of travel of the droplet spray. The nebulizer may be a concentric pneumatic nebulizer. The spray chamber may be substantially V-shaped.

In accordance with a further inventive aspect, a system includes a nebulizer. The nebulizer includes a droplet spray source for producing a droplet spray from a liquid sample stream and a gas stream and a spray chamber having an upper portion, a central portion, and a lower portion. The droplet spray source introduces the droplet spray into the central portion of the spray chamber from an entry point in a primary horizontal direction of travel towards a wall of the central portion of the spray chamber. The upper portion is positioned above the entry point and is connected to the central portion of the spray chamber to receive gas flow and entrained droplets flowing from the central portion. The lower portion is positioned below the entry point and is connected to the central portion of the spray chamber for receiving accumulated liquid in the spray chamber. The system includes an exit positioned in the upper chamber at a position that is horizontally offset relative to the entry point in primary horizontal direction of travel for gas flow and entrained droplets to leave the spray chamber. The system further includes a detector for detecting components of the sample that were in the droplets.

The system may include a desolvator for receiving the droplets exiting the spray chamber via the exit, desolvating the exiting droplets to produce analyte particles, and passing the analyte particles onto the detector. The detector may be a charged aerosol detector. The wall in the central portion of the spray chamber to which the droplet spray may be directed to be perpendicular to the primary horizontal direction of travel. The wall in the central portion of the spray chamber to which the droplet spray is directed may be oriented at an obtuse angle relative to the primary horizontal direction of travel. The nebulizer may be a concentric pneumatic nebulizer.

### BRIEF DESCRIPTION OF THE DRAWINGS

FIG. 1 depicts a cross-sectional view of an illustrative nebulizer spray chamber assembly.

FIG. 2 depicts a cross-sectional view of a nebulizer spray chamber assembly of an exemplary embodiment.

FIG. 3 depicts a cross-sectional view of an illustrative nozzle tip for a nebulizer spray chamber assembly of an exemplary embodiment.

FIG. 4 depicts a cross-sectional view of a nebulizer spray chamber assembly of another exemplary embodiment.

FIG. 5 depicts a block diagram of an illustrative CAD system of an exemplary embodiment.

FIG. 6A depicts a table of experimental data for a CAD system that used an illustrative nebulizer spray chamber assembly design like that of FIG. 1.

FIG. 6B depicts a plot of sugar levels detected for various concentrations of solutions for the experiment for a CAD system that used an illustrative nebulizer spray chamber assembly design like that of FIG. 1.

FIG. 7A depicts a table of experimental data for a CAD system that used an inventive nebulizer spray chamber assembly design such as described herein.

FIG. 7B depicts a plot of sugar levels detected for various concentrations of solutions for the experiment for a CAD system that used the inventive nebulizer spray chamber assembly design described herein.

### DETAILED DESCRIPTION

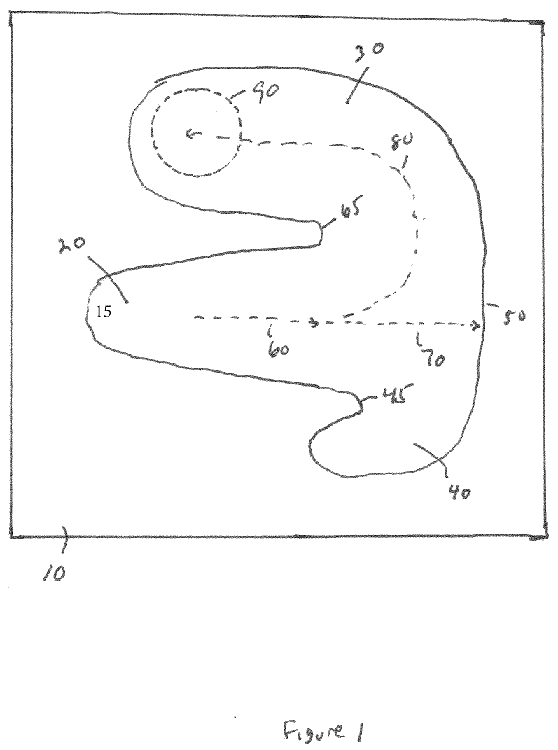

FIG. 1 depicts a nebulizer spray chamber assembly 10 in which a nebulizer sprays a droplet spray of a liquid sample. The nebulizer spray chamber assembly 10 is divided into a central region 20, a lower region 40, and an upper region 30. The lower region 40 is partially divided from the central region 20 by means of a horizontally projecting rib or partition 45. The upper region 30 is partially divided from the central region 20 by a horizontally projecting rib or partition 65.

As shown in FIG. 1, droplets formed in an emitter nozzle travel principally in a horizontal direction as indicated by dotted line 60 toward wall 50. Droplets of a sufficiently small size are entrained by gas flow and pass into the upper region 30. The gas flow and the entrained droplets negotiate around horizontally projecting rib or partition 65 and travel through the upper region 30 from right to left as indicated by dotted line 80 and then exit through exit port 90. Exit port 90 communicates with a charging chamber via a desolvation tube or desolvator, where the nonvolatile residue charging particles may be electrically charged for subsequent detection.

The wall 150 is curved in the upper region 30 to assist in guiding the gas flow to the exit 90 while maintaining a smooth flow of gas in the upper region without turbulent flow patterns or eddies. The wall 50 in the medial portion of the central region 20 is straight and oriented substantially parallel to an entry point 15 (e.g., the aperture of a nozzle), and the wall 50 is oriented substantially perpendicular to the central axis of a droplet plume emitted from the emitter (see dotted line 70). Larger droplets in the droplet spray impact the medial portion of the wall 50. The lower portion of wall 50 is curved so that liquid accumulating on the wall 50 flows downward under the influence of gravity into the lower region 40, where the accumulated liquid may be removed by a drain port to waste.

One disadvantage of the spray chamber design shown in FIG. 1 is that droplets may settle on the upper surface of the horizontally projecting rib or partition 65. Such droplets may migrate down the slope of the upper surface of the horizontally extending rib or partition 65 and fall off. The falling droplets get entrained in the plume of more recently emitted droplets from the emitter, impact the wall 50, and form smaller droplets that may then exit the spray chamber via exit port 90. The result may be band broadening that may obscure the presence of a second analyte peak of low intensity at a detector.

The nebulizer spray chamber assembly of exemplary embodiments may eliminate the horizontally extending rib or partition 65. This may avoid the droplets falling off the horizontally extending rib or projection 65 into the droplet spray plume and thus may eliminate or reduce such band broadening.

The elimination of the horizontally extending rib or partition 65 in the exemplary embodiments also may help eliminate the reversal of flow of gas and entrained droplets and allow the gas flow and entrained droplets in a spray chamber to flow in the primary horizontal direction of droplet spray from an emitter, like a nozzle of a nebulizer. The term “primary” is used to specify this direction as the droplets are emitted and flow primarily in the specified primary horizontal direction but some droplets may modify their direction due to collisions, striking a wall, currents in the chamber, etc. Moreover, since the plume is roughly conical, flow of portions of the conical plume tend to diverge from the primary horizontal direction somewhat as the droplets travel and the diameter of the plume cone increases. The elimination of the reversal of gas and entrained droplets flow may be further aided by removing wall surfaces that encourage such reversal of flow. As a result, the exemplary embodiments may produce better data as more analyte may be detected than with the spray chamber design of FIG. 1. Specifically, it is believed that the reversal of gas and entrained droplet flow in such a design may cause some desirable large droplets from being transported to the evaporation tube, desolvated, and detected. As a result, there may be a reduction in the signal detected due to the reversal of gas and entrained droplets flow. This can negatively impact the limits of quantitation and detection.

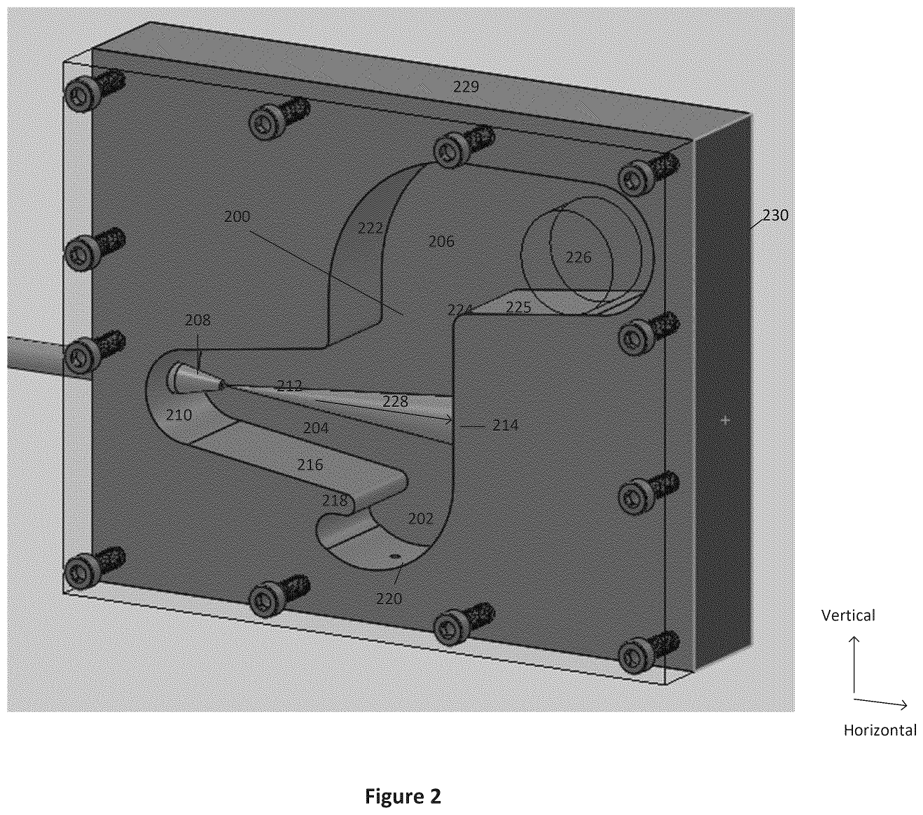

FIG. 2 depicts a nebulizer spray chamber assembly 200 in accordance with an exemplary embodiment. The nebulizer spray chamber assembly 200 may be formed from an inert, corrosion resistant sturdy material, such as a plastic, polytetrafluoroethylene, polypropylene, a ceramic, steel, or the like. The nebulizer spray chamber assembly 200 may include a spray chamber with a lower portion 202, a central portion 204, and an upper portion 206. A nozzle 208 of an emitter for a nebulizer may be secured to a wall 210 in the central portion 204 of the spray chamber. The nebulizer may be, for example, a concentric pneumatic nebulizer, a parallel path nebulizer, or the like. The nozzle 208 may emit a plume 212 of droplet spray that includes droplets and gas. The plume 212 may be emitted from an entry point (such as an aperture of the nozzle 208) in a direction that is perpendicular to the wall 214. The wall 214 may be a straight surface oriented roughly vertically in the central portion 204 in this exemplary embodiment. The wall 210 to which the nozzle 208 is secured may be curved to facilitate the flow of any droplets that accumulate on the wall 210. The flow may travel down downward sloping surface 216. A projection 218 is provided. The flow of droplets may fall off the rounded lip of the projection 218 into the lower portion 202 of the spray chamber.

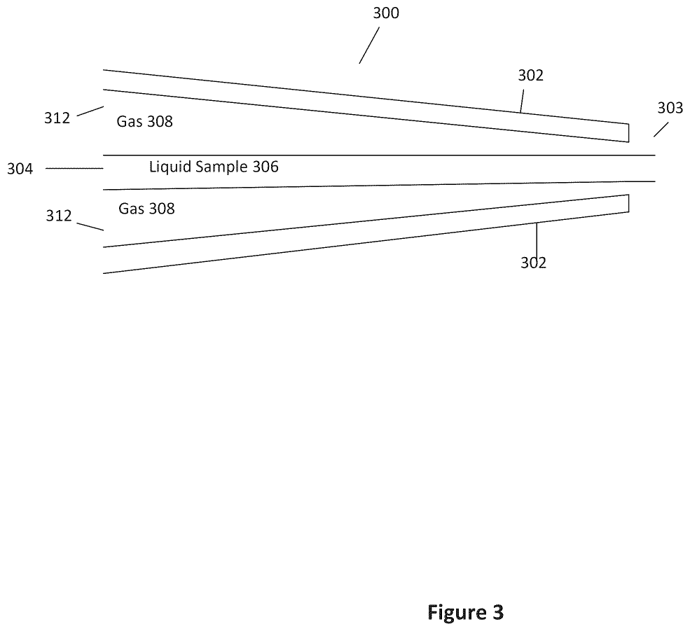

FIG. 3 depicts a nozzle tip 300, such as the tip of nozzle 208 for a nebulizer, such as a concentric pneumatic nebulizer, that may be used in exemplary embodiments. The nozzle tip 300 includes an outer housing 302 that may form a cone leading to an aperture 303. The housing 302 may be formed of a suitable material, such as steel, that is sufficiently non-reactive and that possesses enough tensile strength to be durable and to hold its shape. An inner conduit 304 may be cylindrically shaped and positioned centrally within the cone formed by the housing 302. The inner conduit 304 may contain a liquid sample stream 306, such as eluate from a liquid chromatography column, that contains both one or more solvents and a sample. The space 312 between the housing 302 and the inner conduit 304 may serve as a conduit for gas 308 from a gas source. This gas 308 is used to form an aerosol by outputting the gas 308 under pressure at the aperture 303 along with the liquid sample 306 stream. The gas 308 and liquid sample 306 stream combine to produce an aerosol in plume 212.

As can be seen in FIG. 2, the lower portion of the spray chamber 200 may contain a drain port 220 for draining accumulated liquid from droplets from the spray chamber 200 to waste. The curve of the lower portion 202 helps to directed accumulated liquid to the drain port 220.

The upper portion 206 of the spray chamber 200 may include wall 222. The wall 222 may be curved to help direct gas flow and entrained droplets to exit 226. The curved wall 222 may be smooth to help ensure that there is smooth non-turbulent gas flow. Transition 224 at the top of wall 214 may be curved as well to ensure smooth non-turbulent flow toward exit 226. The transition 224 leads to a slightly downward sloped surface 225 that leads to the exit 226. The slightly downward slope may help with draining of droplets to the drain port 220.

Arrow 228 indicates the primary direction of the plume 212 as emitted from the aperture of the nozzle 208 (“the entry point”). This primary direction as indicated by arrow 228 may be substantially perpendicular to wall 214. The resulting gas flow may entrain droplets that rise together into the upper portion 206 of the spray chamber. The gas and entrained droplets then may leave the nebulizer spray chamber assembly 200 via exit 226. The exit 226 may be oriented substantially in the direction of arrow 228 but vertically above (see Vertical axis) relative to the nozzle 208.

The exit 226 may be located at different positions in some embodiments. For example, nebulizer spray chamber assembly 200 may have exit 226 located in either the upper surface 229 or the side surface 230. Moreover, the exit 226 may be located in a more horizontally displaced location that is displaced toward the nozzle 208.

In this embodiment, there is no horizontally (See Horizontal axis) extending rib or partition. As such, this embodiment is not troubled with the problems caused by droplets dropping from the horizontally extending rib or partition into the plume 212. Further, this embodiment does not reverse the gas flow and as a result, may detect more analyte than the prior art spray chamber design discussed above.

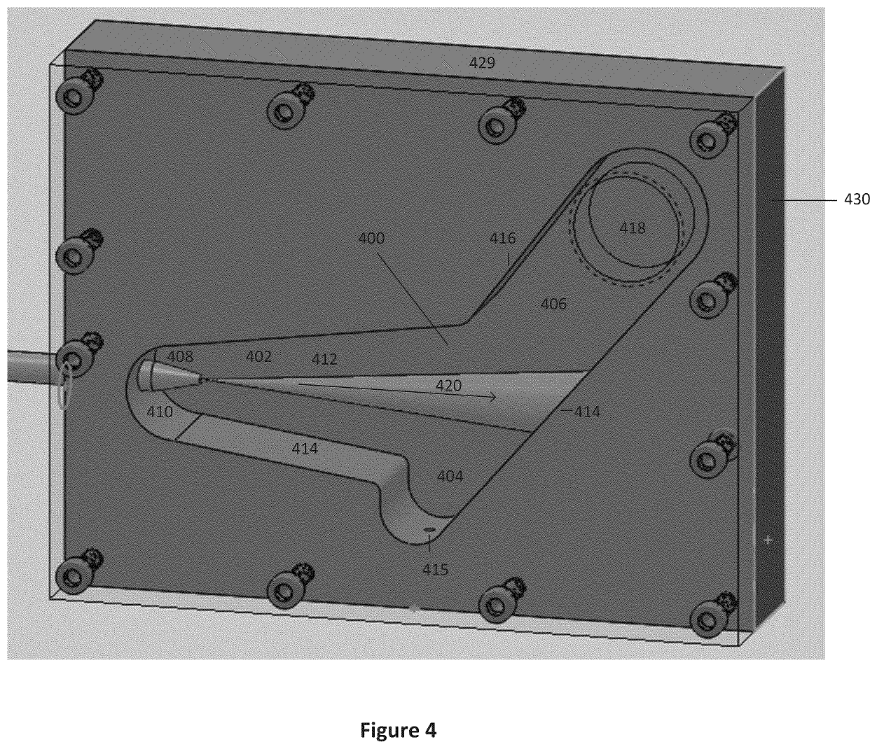

FIG. 4 depicts another exemplary embodiment of a nebulizer spray chamber assembly 400. The spray chamber shown is partitioned into an upper portion 406, an central portion 402, and a lower portion 404. The spray chamber is similar in many respects to the spray chamber of FIG. 2. For instance, there is a nebulizer nozzle 408 connected to curved wall 410. The nozzle 408 generates a plume 412 directed in direction 420. A downward sloping surface leads to the lower portion 404, which contains a drain port 415.

The spray chamber differs from the spray chamber of FIG. 2 in several respects. The wall 414 is angled at an obtuse angle relative to the aperture of the nozzle 408 as opposed to being oriented substantially parallel to the aperture of the nozzle 408. This obtuse angle helps to direct the gas and entrained droplets upward in the upper portion and toward exit 418. The wall 416 of the upper portion 406 is not curved like the wall 222; rather wall 416 is straight and is positioned at an obtuse angle relative to the aperture of the nozzle. This orientation of wall 416 helps guide gas and entrained droplets to the exit 418. The exit 418 is positioned in the same direction 420 as the plume 412 is emitted from nozzle 408 (see arrow 420) and is oriented vertically above the nozzle 408. Nebulizer spray chamber assembly 400 may be designed to have exit 418 be located in either the upper surface 429 or side surface 430 in some exemplary embodiments.

The nebulizer spray chamber assembly 400 does not have a horizontally extending rib or partition that may cause droplets to enter the plume 420. In addition, like the nebulizer spray chamber assembly 200 of FIG. 2, nebulizer spray chamber assembly 400 does not reverse the direction of the gas flow.

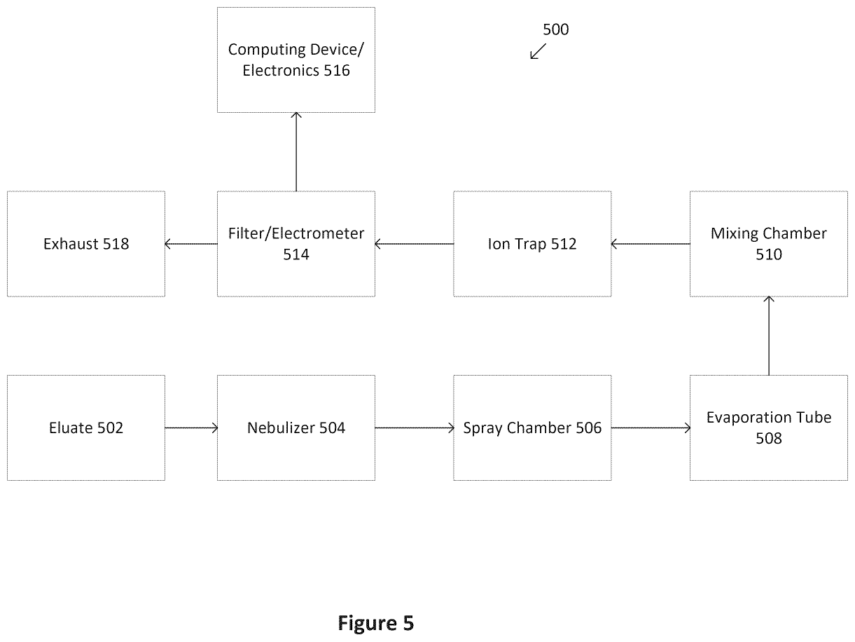

The nebulizer spray chamber assemblies 200 and 400 may be used in a variety of different applications. For instance, they may be used in CADs and other types of detectors. FIG. 5 depicts a block diagram 500 of a CAD system in which a spray chamber, like the spray chambers 200 and 400, may be incorporated. A liquid chromatography column may pass a sample that was introduced into a mobile phase that flows through a stationary phase in a chromatography column. The resultant eluate 502 exiting the liquid chromatography column may be passed to a nebulizer 504, such as a concentric pneumatic nebulizer. The nebulizer 504 nebulizes the liquid stream of eluate 502 to generate an aerosol plume in the spray chamber 506 as described above. Gas with entrained droplets in the plume exit the spray chamber 506 and enter an evaporation tube 508. The evaporation tube 508 may be heated to encourage evaporation. The resultant aerosol residue particles enter a mixing chamber 510 where they may be charged by an ion jet formed via corona discharge. In the mixing chamber 510, the dried residue particles mix with charged particles, resulting in transfer of charge to the dried residue particles. The charged aerosol residue particles may then pass to an ion trap 512. The ion trap 512 may remove excess ions and high mobility charged particles. Specifically, excess nitrogen and water ions may be removed. The remaining charged particles are collected by an electrode, such as an electrically conductive filter. The collection of these dried analyte particles is registered by an electrometer (see filter/electrometer 514). The output signal from the filter/electrometer 514 may indicate the concentration of analytes, and the output signal may be processed and associated data may be displayed by a computing device or other electronics 516. The gas may them be exhausted via a gas exhaust 518.

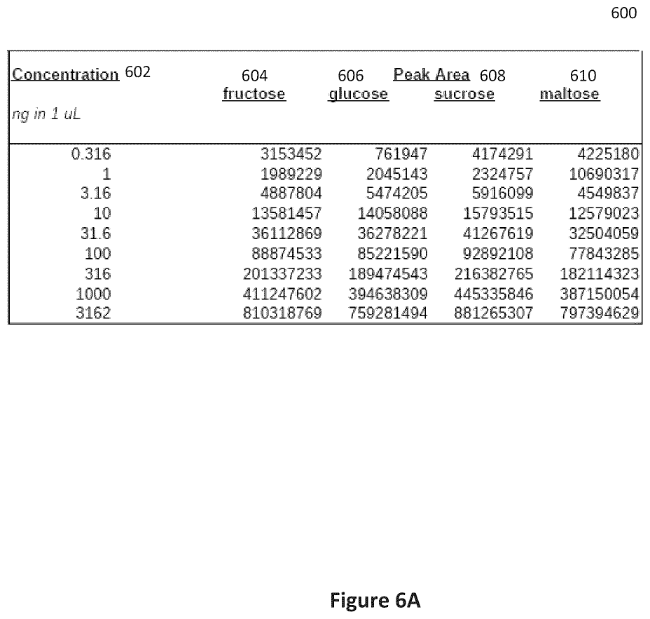

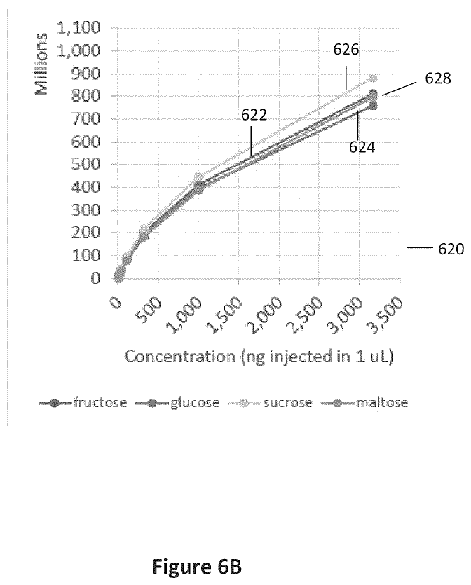

An experiment was conducted wherein a sequence of 1 microliter injections of solutions containing increasing concentrations of four sugars: fructose, glucose, sucrose, and maltose, were passed through a liquid chromatography column and the eluate was nebulized and detected by a CAD system. The separation was performed using a mobile phase of 25% water and 75% acetonitrile flowing at 0.3 ml/min using a same chromatography column. First, a CAD with a spray chamber like that of FIG. 1 was used to perform the experiment. FIG. 6A depicts a table 600 of results for this instance of the experiment. The table 600 includes the concentration 602 of the solution, the detected peak areas for fructose 604, glucose 606, sucrose 608, and maltose 610. Each row is associated with a specific concentration of solution. FIG. 6B depicts a corresponding plot 620 of detected amounts of the respective sugars relative to solution concentration. Plot line 622 is for fructose, plot line 624 is for glucose, plot line 626 is for sucrose, and plot line 628 is for maltose.

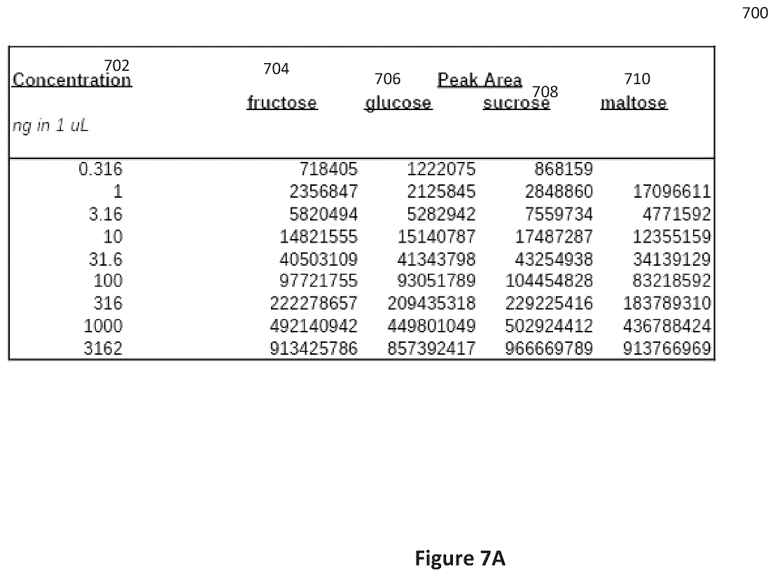

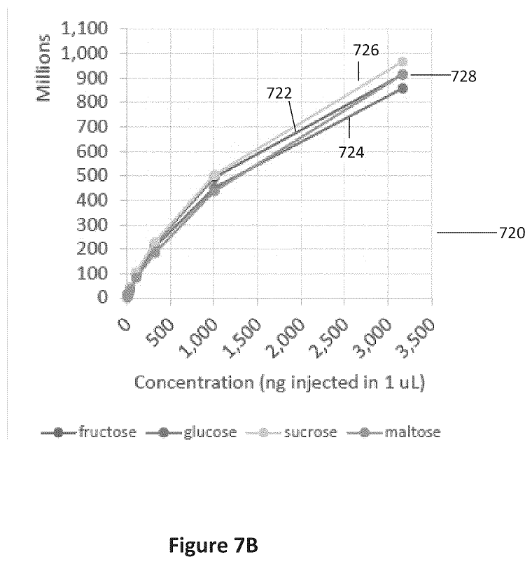

The experiment then was performed on a CAD system that used the spray chamber design of FIG. 2. FIG. 7A depicts a table 700 showing solution concentration 702, peak area for fructose 704, glucose 706, sucrose 708 and maltose 710. Each row holds data for a specific solution concentration. As can be seen, the peak areas for the sugars are higher for the CAD system using the spray chamber designs of the exemplary embodiments. This likely results from there being no reversal of flow and consequent loss of desirable larger droplets.

FIG. 7B, shows a plot 700 of detected levels of the four sugars for increasing solution concentration levels. Plot line 722 is for fructose, plot line 724 is for glucose, plot line 726 is for sucrose, and plot line 728 is for maltose. Comparing plot 720 with plot 620 reveals that higher levels of the four sugars are detected when the inventive spray chamber design of an exemplary embodiment is used. This should result in improved limits of quantitation and detection compared to data generated when using a spray chamber as depicted in FIG. 1.

While exemplary embodiments have been described herein, various changes in form and detail may be made without departing from the intended scope of the appended claims and equivalents thereof.

1. A nebulizer and spray chamber assembly, comprising:
a nebulizer for producing a droplet spray from a liquid sample stream and a gas stream;
a spray chamber having an upper portion, a central portion and a lower portion,
the nebulizer introducing the droplet spray into the central portion of the spray chamber from an entry point in a primary horizontal direction of travel towards a wall of the central portion of the spray chamber that is substantially perpendicular to the primary horizontal direction of travel of the droplet spray,
the upper portion being positioned above the entry point and being connected to the central portion of the spray chamber to receive gas flow and entrained droplets flowing from the central portion, and
the lower portion being positioned below the entry point and being connected to the central portion of the spray chamber for receiving accumulated liquid in the spray chamber; and
an exit positioned in the upper chamber at a position, that is horizontally offset relative to the entry point in the primary horizontal direction of travel, for the gas flow and entrained droplets to leave the spray chamber.

2. The nebulizer and spray chamber assembly of claim 1, wherein the nebulizer comprises an emitter nozzle.

3. The nebulizer and spray chamber assembly of claim 2, wherein the emitter nozzle is connected to a wall of the central portion of the spray chamber.

4. The nebulizer and spray chamber assembly of claim 1, further comprising a drain positioned in the lower portion of the spray chamber for draining the accumulated liquid.

5. The nebulizer and spray chamber assembly of claim 4, wherein the lower portion of the spray chamber includes a curved wall for directing the accumulated liquid to the drain.

6. The nebulizer and spray chamber assembly of claim 1, wherein the upper portion of the spray chamber includes a curved wall for directing the gas flow and entrained droplets to the exit.

7. The nebulizer and spray chamber assembly of claim 1, wherein the nebulizer is a concentric pneumatic nebulizer.

8. A nebulizer and spray chamber assembly, comprising:
a nebulizer for producing a droplet spray from a liquid sample stream and a gas stream;
a spray chamber having an upper portion, a central portion and a lower portion,
the nebulizer introducing the droplet spray into the central portion of the spray chamber from an entry point in a primary horizontal direction of travel towards a wall of the central portion of the spray chamber that is oriented at an obtuse angle relative to the primary horizontal direction of travel of the droplet spray,
the upper portion being positioned above the entry point and being connected to the central portion of the spray chamber to receive gas flow and entrained droplets flowing from the central portion, and
the lower portion being positioned below the entry point and being connected to the central portion of the spray chamber for receiving accumulated liquid in the spray chamber; and
an exit positioned in the upper chamber at a position, that is horizontally offset relative to the entry point in primary horizontal direction of travel, for the gas flow and entrained droplets to leave the spray chamber.

9. The nebulizer and spray chamber assembly of claim 8, wherein the droplet spray source comprises an emitter nozzle.

10. The nebulizer and spray chamber assembly of claim 9, wherein the emitter nozzle is connected to a wall of the central portion of the spray chamber.

11. The nebulizer and spray chamber assembly of claim 8, further comprising a drain positioned in the lower portion of the spray chamber for draining the accumulated liquid.

12. The nebulizer and spray chamber assembly of claim 8, wherein the upper portion of the spray chamber includes an extension of the wall of the central portion of the spray chamber that is at the obtuse angle relative to the primary horizontal direction of travel of the droplet spray.

13. The nebulizer and spray chamber assembly of claim 8, wherein the nebulizer is a concentric pneumatic nebulizer.

14. The nebulizer and spray chamber assembly of claim 8, wherein the spray chamber is substantially V-shaped in profile.

15. A system, comprising:
a nebulizer, comprising:
a droplet spray source for producing a droplet spray from a liquid sample stream and a gas stream;
a spray chamber having an upper portion, a central portion and a lower portion,
the droplet spray source introducing the droplet spray into the central portion of the spray chamber from an entry point in a primary horizontal direction of travel towards a wall of the central portion of the spray chamber,
the upper portion being positioned above the entry point and being connected to the central portion of the spray chamber to receive gas flow and entrained droplets flowing from the central portion, and
the lower portion being positioned below the entry point and being connected to the central portion of the spray chamber for receiving accumulated liquid in the spray chamber;
an exit positioned in the upper chamber at a position, that is horizontally offset relative to the entry point in the primary horizontal direction of travel, for gas flow and entrained droplets to leave the spray chamber; and
a detector for detecting components of the sample that were in the droplets.

16. The system of claim 15, further comprising a desolvator for receiving the droplets exiting the nebulizer via the exit and desolvating the exiting droplet to produce analytes and passing the analytes onto the detector.

17. The system of claim 15, wherein the detector is a charged aerosol detector.

18. The system of claim 15, wherein the wall in the central portion of the spray chamber to which the droplet spray is directed is perpendicular to the primary horizontal direction of travel.

19. The system of claim 15, wherein the wall in the central portion of the spray chamber to which the droplet spray is directed is oriented at an obtuse angle relative to the primary horizontal direction of travel.

20. The system of claim 15, wherein the nebulizer is a concentric pneumatic nebulizer.
